(19) United States
(12) Patent Application Publication
(10) Pub. No.: US 2008/0249310 A1
(43) Pub. Date: Oct. 9, 2008

(54) Process For the Preparation of (2R,3R)-2-Hydroxy-3-Amino-3-Aryl-Propionamide and (2R,3R)-2-Hydroxy-3-Amino-3-Aryl-Propionic Acid Alkyl Ester

(76) Inventors: Thijs Kuilman, Venlo (NL); Horst Straatman, Horst (NL)

Correspondence Address: NIXON & VANDERHYE, PC, 901 NORTH GLEBE ROAD, 11TH FLOOR, ARLINGTON, VA, 22203, US

(21) Appl. No.: 11/658,156

(22) PCT Filed: Jul. 20, 2005
(86) PCT No.: PCT/EP05/07986

§ 371 Date: Feb. 14, 2008

(30) Foreign Application Priority Data
Jul. 23, 2004 (EP) 04077142.0

(51) Int. Cl. C07D 471/14; C07C 231/02

(52) U.S. Cl. 546/82; 564/134

(57) ABSTRACT

The invention relates to a novel compound (2R,3R)-2-hydroxy-3-amino-3-aryl-propionamide according to formula (1), wherein aryl A represents a substituted or unsubstituted aromatic ring,

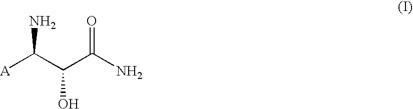

and to a process for the preparation of said compound of formula (1), wherein a reaction mixture comprising the two enantiomers (2R,3S) and (2S,3R) of trans-3-aryl glycidic acid alkyl ester and the two enantiomers (2R,3R) and (2S,3S) of cis-3-aryl glycidic acid alkyl ester, said mixture being enantiomerically and diastereomerically enriched in the (2R,3S)-trans-3-arylglycidic acid alkyl ester, is reacted with ammonia. The invention further relates to a process for the preparation of (2R,3R)-2-hydroxy-3 amino-3-aryl-propionic alkyl ester.

The invention relates to a process for the preparation of (2R,3R)-2-hydroxy-3-amino-3-aryl-propionamide according to formula (1), wherein the aryl A may be a substituted or unsubstituted aromatic ring,

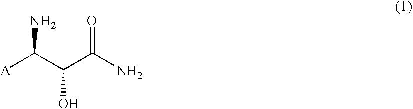

in which a reaction mixture comprising the two enantiomers (2R,3S) and (2S,3R) of trans-3-aryl glycidic acid alkyl ester and the two enantiomers (2R,3R) and (2S,3S) of cis-3-aryl glycidic acid alkyl ester, said ester being represented by the general formula (2), wherein R¹ is an ester residue which may be an optionally substituted alkyl, cycloalkyl, aryl, aralkyl or alkaryl group,

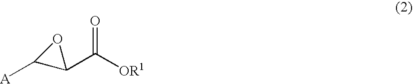

is reacted with ammonia.

In Zhou et al. Synth. Commun. 2003, Vol. 33, No.5, p.723-728, an ammonolysis reaction is disclosed of racemic trans-3-phenylglycidic acid ethyl ester (defined as trans-epoxide) into racemic 2-hydroxy-3-amino-3-phenyl-propionamide (defined as racemic phenyl isoserine amide) as the major product.

In Jacobsen et al. J. Org. Chem. 1992, 57, 4320-4323 a process is described for the preparation of (2R,3S)-2-hydroxy-3-amino-3-phenyl-propionamide (defined as (2R,3S)-phenyl isoserinamide) by dissolving (2R,3R)-ethyl-3-phenyl glycidate in a solution of ethanol saturated with ammonia. The (2R,3R)-ethyl-3-phenyl glycidate is obtained from a mixture comprising a 10:1 cis:trans ratio of ethyl-3-phenyl glycidate, wherein the e.e. of the (2R,3R)-cis-ethyl-3-phenyl glycidate is between 95 and 97%. Diastereomerically pure product (2R,3S)-phenyl isoserinamide could be isolated by recrystallization from methanol.

None of these processes describe how to prepare the compound (1) of the present invention in acceptably/relatively high enantiomeric (e.e.) and diastereomeric (d.e.) purity.

It is the object of the present invention to prepare compound (2R,3R)-2-hydroxy-3-amino-3-aryl-propionamide of formula (1) in relatively high yield and in relatively high e.e. and d.e.

According to the invention this can be achieved by using a reaction mixture that is enantiomerically and diastereomerically enriched in the (2R,3S)-trans-3-aryl-glycidic acid alkyl ester. (2R,3S)-trans-3-aryl-glycidic acid alkyl ester can be represented by formula (2a) below.

Surprisingly, the optically pure compound (1) can be easily isolated in relatively high yield, d.e. and e.e. It is very advantageous that little or no further purification or recrystallization is required. It is a further advantage that an economically attractive process can be applied on an industrial scale.

The invention also relates to the use of the optically active compound according to formula (1) as an intermediate in the preparation of pharmaceuticals, and thus, in the framework of the present invention, it is an object to prepare the compound according to formula (1) in relatively high stereoisomeric purity.

The invention further relates to a novel compound (2R,3R)-2-hydroxy-3-amino-3-aryl-propionamide according to formula (1), wherein aryl A may be a substituted or unsubstituted aromatic ring, for instance a phenyl ring, i.e. (2R,3R)-2-hydroxy-3-amino-3-phenyl-propionamide.

The invention further relates to a process for the preparation of (2R, 3R)-2-hydroxy-3-amino-3-aryl-propionic acid alkyl ester according to formula (3)

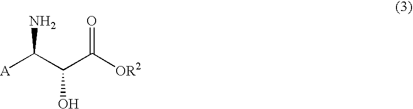

wherein R2 may in principle be any alkyl ester residue as defined for R¹ above. Preferably, R² is a substituted or unsubstituted alkyl group with 1-10 carbon atoms, more preferably 1-6 carbon atoms. Preferred examples of R²groups are methyl, ethyl, propyl, n-butyl, isopropyl, isobutyl, tert-butyl, or the like.

WO 03/003804 in the name of Altana Pharma AG describes a process for preparing a (2R,3R)-2-hydroxy-3-amino-3-phenyl-propionic acid ethyl ester (defined as (2R,3R)-3-phenylisoserine ethyl ester) by reacting the racemic mixture of 3-phenylisoserine ethyl ester with L-(+)-tartaric acid as optical resolving agent.

### Definitions

The following terms are used interchangeably throughout the description of the present invention: 3-aryl isoserine amide or 2-hydroxy-3-amino-3-arylproprionamide; 3-aryl serine amide or 2-amino-3-hydroxy-3-arylproprionamide; cis-3-arylglycidic acid alkyl ester or cis-aryl epoxide or cis-3-aryl-glycidate; trans-3-arylglycidic acid alkyl ester or trans-aryl epoxide or trans-3-aryl-glycidate.

A reaction mixture comprising the enantiomers of compound (2) according to formulae (2a) to (2d) as shown below is a crude reaction mixture which, after the synthesis of compound (2), has not necessarily undergone any additional purification or separation step and thus may contain components other than the enantiomers of compound (2). The reaction mixture may comprise a solvent. Preferably, the reaction mixture comprises only a very small amount of the cis-enantiomers of compound (2) according to formulae (2c) and (2d) as shown below.

The enantiomeric excess e.e. is equal to the difference between the amounts of enantiomers divided by the sum of the amounts of the enantiomers, which quotient can be expressed as a percentage after multiplication by 100.

The diastereomeric excess d.e. is equal to the difference between the amounts of diastereomers divided by the sum of the amounts of the diastereomers, which quotient can be expressed as a percentage after multiplication by 100.

### DETAILED DESCRIPTION OF THE INVENTION

### Compound (2)

The process of the present invention for the preparation of (2R,3R)-2-hydroxy-3-amino-3-aryl-propionamide according to formula (1) comprises reacting a reaction mixture comprising compound (2) with ammonia wherein said reaction mixture comprises the two enantiomers (2R,3S) and (2S,3R) of trans-3-arylglycidic acid alkyl ester and the two enantiomers (2R,3R) and (2S,3S) of cis-3-arylglycidic acid alkyl ester of formula (2), said mixture being enantiomerically and diastereomerically enriched in the (2R,3S)-trans-3-arylglycidic acid alkyl ester.

The process may be applied in an organic solvent, preferably a water soluble solvent. Suitable solvents may be alcohols, for example methanol, ethanol and the like, or ethers, for example tetrahydrofuran and the like. Preferably, the process is applied in water, more preferably in a mixture of water and a solvent, and even more preferably in a mixture of water and an alcohol.

The alkyl esters of trans-3-arylglycidic acid according to formula (2) have two chiral centres. Molecules with n chiral centres have 2ⁿ stereoisomers. For 3-arylglycidic acid alkyl esters, therefore, four stereoisomers are conceivable, which occur as two D, L pairs, which are mutually diastereoisomers. The two diastereoisomeric forms of 3-arylglycidic acid alkyl esters are the cis and the trans form. The two enantiomers in the trans form have (2R,3S) and (2S,3R) as configuration, as shown by formulae (2a) and (2b) respectively. The configurations of the cis form are (2R,3R) and (2S,3S), as shown by formulae (2c) and (2d) respectively.

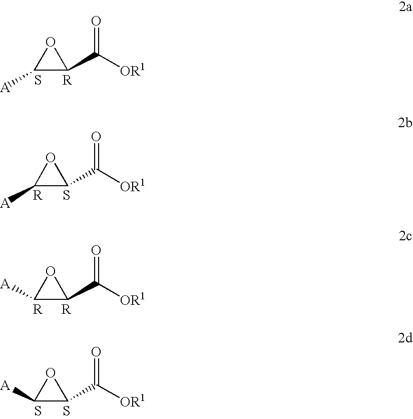

Preferably, the reaction mixture of the present invention contains only a very small amount of the cis-enantiomers (2c) and (2d).

The 3-arylglycidic acid alkyl ester according to formula (2) can optionally contain one or more substituents on the aromatic ring A. The aromatic ring A may be mono- or polycyclic. The aromatic ring may also be heteroaromatic, for example pyridine, quinoline, and the like. One or more substituents may be present at one or more of the 2-, 3- or 4-position of the aryl, preferably at the 3- and/or 4-position, more preferably at the 4-position. The aryl group may contain one substituent or may contain two or more substituents. If it contains more than one substituent, the substituents may be the same or different. If there are two substituents on the aryl, the aryl is preferably 3-,4-disubstituted or 3-,5-disubstituted. If there are three substituents on the aryl, the aryl is preferably 3-, 4-, 5-trisubstituted.The substituents may be chosen, for instance, from hydroxy; alkoxy with 1-6 C atoms, for example methoxy, ethoxy, butoxy and the like; acetoxy R—(C═O)O—; alkyl with 1-6 C atoms; halogen, NH₂, NO₂ and nitrile CN. If the substituent is a hydroxy or NH₂-group, said hydroxy or NH₂-group may be protected during the process of the invention by using standard protection groups which are commonly known in the art, such as acyl groups, silyl groups, oxycarbonyl groups such as tert-butyl-oxycarbonyl (BOC) and the like.

R¹ in formula (2) is a group derived from an alcohol and is an ester residue. R¹ may be a chiral or achiral group. R¹ in formula (2) may represent an optionally substituted alkyl; cycloalkyl, for example menthyl, chloro acetic acid menthyl, and the like; aryl, for example phenyl; aralkyl, for example benzyl, phenylethyl and the like, or alkaryl. Preferably R¹ is a substituted or unsubstituted alkyl group having 1-20 C atoms, preferably 2-10 C atoms, and is advantageously an alkyl group with 1-6 carbon atoms; preferably R¹ is methyl, ethyl, propyl, isopropyl, isobutyl, or tert-butyl, more preferably ethyl.

According to the process of the invention, the reaction mixture is enantiomerically enriched in the (2R,3S)-trans-3-arylglycidic acid alkyl ester relative to the (2S,3R)-trans-3-arylglycidic acid alkyl ester, for example in an e.e. of at least about 40%, preferably at least about 50%, more preferably at least about 60%, even more preferably at least about 70% and most preferably at least about 80%. According to a further preferred embodiment of the present invention, crystallization of the compound of formula (1) may occur by seeding the reaction mixture with small quantities of optically pure compound (1).

According to the process of the invention, the reaction mixture is diastereomerically enriched in the trans-3-arylglycidic acid alkyl ester relative to the cis-3-arylglycidic acid alkyl ester, for example in a d.e. of at least about 30%, preferably at least about 50% by weight of trans, more preferably at least about 70% by weight, even more preferably at least about 90% by weight and most preferably at least about 95% by weight. It is especially preferred for the reaction mixture to contain only a small amount of cis-3-arylglycidic acid alkyl ester. The reaction mixture may also be basically diastereomerically pure in the trans-3-arylglycidic acid alkyl ester.

The amount of cis-ester according to formulae (2c) and (2d) relative to the weight of the required trans-ester (2R, 3S)-trans-3-arylglycidic acid alkyl ester according to formula (2a), may be higher than about 3%, or even higher than about 5%, or even higher than about 10%, but preferably lower than about 45%, more preferably lower than about 35%, even more preferably lower than about 25%, and most preferably lower than about 15%.

In the process of the invention, the enantiomerically and diastereomerically enriched reaction mixture of 3-aryl-glycidic acid alkyl ester according to formula (2) is reacted with ammonia, preferably in a protic solvent, more preferably in water. Preferably, the ammonia concentration is between about 15 to 35% by weight, more preferably between about 20 and 30% by weight, most preferably between about 22 and 25% by weight.

The reaction may be carried out at a temperature of between about 0 and 75° C., preferably at a temperature of between about 15 to 65° C., in particular at a temperature of between about 20 to 40° C.

The reaction may be carried out under atmospheric pressure, or in a closed vessel under pressure. In the case of a closed vessel, the ammonia concentration may be higher than about 35%.

This reaction can, for example, be carried out according to known processes, such as exemplified in Wuts et.al. Tetr.: Asymm. 2000, 11, 2117-2123.

When the process is carried out in an organic solvent, the ammonia is usually in the form of NH₃. When the process is carried out in the presence of water, with or without the presence of an organic solvent, the ammonia may be in the form of a mixture of NH₃ and NH₄OH.

In addition to the four enantiomers of the 3-arylglycidic acid alkyl ester of formula (2), the reaction mixture in the process of the present invention may comprise by-products, for example by-products obtained during the preparation of compound (2). The reaction mixture may comprise a solvent. The process for preparing (2R,3R)-2-hydroxy-3-amino-3-aryl-propionamide according to formula (1) may, for example, be carried out in the same solvent as that in which the reaction was carried out to obtain the reaction mixture of compound (2).

The enantiomerically and diastereomerically enriched reaction mixture of compound (2) may, for example, be prepared by stereoselectively hydrolyzing a reaction mixture comprising the two enantiomers (2R,3S) and (2S,3R) of trans-3-arylglycidic acid alkyl ester and the two enantiomers (2R,3R) and (2S,3S) of cis-3-arylglycidic acid alkyl ester of formula (2), by using an enzyme originating from the Candida genus, preferably originating from Candida antarctica. Said enzymatic process is known from EP 0 602 740, which is incorporated herein by reference. Enzymes originating from Candida antarctica are extensively described in WO-8802775. Preferably, the enzyme used according to the subject invention is a lipase originating from Candida antarctica. This lipase can, for instance, be produced via recombinant DNA technology. The gene coding for the lipase in question is heterologously expressed in a host microorganism, for instance Aspergillus oryzae. This enzyme is commercially available, e.g. from NOVO (Novozymes) under the tradename Novozymes® SP 435 and Novozymese SP 525.

When the Candida antarctica lipase enzyme is applied to the reaction mixture comprising compounds according to formulae (2a) to (2d), the enzyme enantioselectively hydrolyses the (2S,3R) enantiomer of the trans-3-phenylglycidic acid alkyl ester with a high selectivity, so that the (2R,3S) ester enantiomer can be obtained with a high e.e. Preferably, the hydrolysed (2S,3R)-trans-3-arylglycidic acid is removed by phase separation via the water phase. The remaining organic phase, comprising at least the non-hydrolysed (2R,3S)-trans-3-arylglycidic acid alkyl ester, and optionally the two cis-esters, and optionally the remaining non-hydrolysed (2S,3R)-trans-3-arylglycidic acid alkyl ester, then represents the reaction mixture for the subsequent step to prepare compound (1). The organic solvent in which the enzymatic reaction is carried out may be partially or fully removed, e.g. by distillation, before using the enantiomerically and diastereomerically enriched reaction mixture in the subsequent step.

According to a preferred embodiment of the present invention, the non-hydrolysed (2R,3S)-trans-ester is not separated from the reaction mixture, and said reaction mixture is used as such in the subsequent step to prepare compound (1).

The stereoselective hydrolysis is preferably effected in a two-phase system comprising an aqueous phase and an organic phase containing an organic solvent. Examples of such a solvent are solvents that are not, or only to a small extent, soluble in water, such as chloroform, isopropyl ether, 3-pentanone, dichloromethane, trichloroethane, benzene, toluene, xylene, methyl t-butyl ether, methyl isobutyl ketone, cyclohexanone, isooctane, ethyl acetate and others. Surprisingly, the selectivity of the enzyme according to the invention is almost independent of the solvent chosen, it being possible in most cases to achieve an e.e. higher than 95% at a conversion below 53%.

The hydrolysis according to the invention can be carried out at room temperature or at elevated temperature. The upper limit is determined by the stability of the substrate and in practice is about 80° C. Preferably, a temperature of 20-60° C. is used, in particular 30-50° C. The use of higher temperatures has the advantage that the reaction proceeds faster.

During the hydrolysis the pH is kept at a value of 5 to 10, preferably 7-9, and in particular at about 8, for instance by adding a base. The residual ester, i.e. the enantiomer that has not been hydrolyzed, can for instance be recovered by separating out the organic solvent in which the ester is dissolved, followed by recovery of the ester from the solution.

Other ways of preparing the reaction mixture of compound (2), which mixture may optionally be enantiomerically and diastereomerically enriched, are described further below (Routes 4 to 6). Said processes according to Routes 4 to 6 may be applied to prepare a reaction mixture comprising the four enantiomers of formulae (2a) to (2d), which reaction mixture may be diastereomerically enriched, enantiomerically and diastereomerically enriched, or may not be enriched at all. A commercially available reaction mixture comprising the four enantiomers of formulae (2a) to (2d) may be applied too.

One way of preparing the reaction mixture may, for example, be by a (chiral) oxidation reaction of an olefin in the presence of a (chiral) catalyst and an oxygen source as oxidation means, for example NaOCI or teit-butylhydroxyperoxide (TBHP), as shown in scheme (4), hereinafter referred to as “Route 4”:

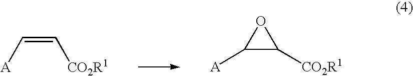

Such a reaction is known, for example, from C. Bonini and G. Righi, Tetrahedron 58 (2002) p 4981-5021. The preferred catalyst is a chiral or asymmetric epoxidation catalyst. In general, the cis (Z)-disubstituted alkenes are the best substrates for the (asymmetric) epoxidation catalysts. Some conjugated disubstituted olefins exhibit isomerisation during the epoxidation reaction, resulting in trans and cis epoxides in different ratios.

A further suitable way of preparing the reaction mixture may be by oxidizing trans (E)-disubstituted alkenes to the corresponding trans epoxides using the catalytic system recently developed by Shi and co-workers, based on a D-fructose (chiral) ketone, as shown by scheme (5), hereinafter referred to as “Route 5”. This Shi reaction is, for example, disclosed in WO 98/15544, which is incorporated herein by reference.

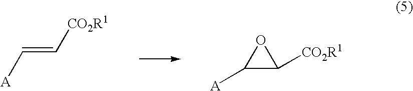

A further interesting route for preparing the reaction mixture may, for example, be by applying the well-known Darzens condensation reaction, in particular by reacting an aromatic aldehyde according to formula (6a),

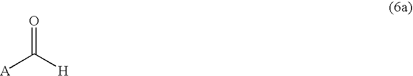

wherein A is as defined above, with an a-halo ester according to formula (6b)

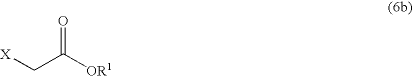

wherein X may be Cl, Br, F or I, and wherein R¹ may be as defined above, in the presence of a base to yield the reaction mixture comprising the two enantiomers (2R,3S) and (2S,3R) of trans-3-arylglycidic acid alkyl ester and the two enantiomers (2R,3R) and (2S,3S) of cis-3-arylglycidic acid alkyl ester of formula (2). This reaction is hereinafter referred to as “Route 6”.

Surprisingly, when Route 6 is applied, the reaction mixture obtained contains a very favourable ratio of trans to cis enantiomers of compound (2). Thus, this Route 6 results in an economically very attractive process.

Any base, organic or inorganic, may be used in the process according to Route 6, but preferably organic bases are used, such as, amines, alkoxides, amides and the like. More preferably, alkoxides of alkaline earth metal M OR³, wherein M is an alkaline earth metal and R³ may in principle be any alkyl group as defined for R¹ above, are used. Preferably, M is selected from Na, K, or Li, preferred alkoxides being methoxides, ethoxides, t-butoxides and the like. Examples of preferred bases are NaOEt, NaOMe, KOEt, KOMe, LiOEt, LiOMe or the like, more preferred bases are NaOEt and NaOMe.

The process according to Route 6 is preferably carried out in the presence of a solvent, more preferably an organic solvent, such as alcohols, for example methanol, ethanol and the like; ethers, such as tetrahydrofuran, t-butylmethylether and the like; optionally aromatic hydrocarbons, such as cyclohexane, toluene, xylene and the like; amides, such as dimethylformamide (DMF) and the like.

In cases where alkoxides are used as the base and in cases where the reaction is carried out in the presence of a solvent, the solvent is preferably the corresponding alkyl alcohol, i.e. the alkylalcohol R³OH having the same R³-substituent as the alkoxide M OR³. Examples of preferred combinations are NaOMe/MeOH, NaOEt/EtOH, KOMe/MeOH, KOEt/EtOH, LiOMe/MeOH, LiOEt/EtOH and the like. The most preferred combination is NaOEt/EtOH.

The process according to Route 6 is preferably carried out using an a-halo ester according to formula (6b) wherein X is Cl and wherein R¹ is as defined above, in particular an ester of chloroacetic acid. Preferably, R¹ is a substituted or unsubstituted alkyl group having 2-10 C atoms, mostly an alkyl group with 1-6 carbon atoms; more preferably R¹ is methyl, ethyl, propyl, isopropyl, isobutyl or tert-butyl or the like. Even more preferably, R¹ is methyl (pref. a-chloro acetic acid methyl ester) or ethyl (pref. or a-chloro acetic acid ethyl ester), and most preferably R¹ is ethyl, since in that case—surprisingly—an optimum in yield in combination with a favourable cis:trans ratio of compound (2) is obtained.

According to a preferred embodiment of the present invention, a combination of an a-halo ester according to formula (6b) with the alkoxide base corresponding with R¹ and the alcohol corresponding with R¹ is used.

Preferred aromatic aldehydes of formula (6a) that may be used in the process of the present invention are benzaldehyde, anisaldehyde and the like, preferably benzaldehyde.

The reagents may generally be added in any order in the process according to Route 6. A preferred way of applying the process according to Route 6 is by charging the base and the aromatic aldehyde (6a) first and then adding the a-halo ester of formula (6b). In that case an optimum in yield in combination with a favourable cis:trans ratio of compound (2) may be obtained.

The Darzens reaction of Route 6 may be carried out at a temperature of between about −20 and +20° C., preferably at a temperature of between about −10 and +10° C., in particular at a temperature of between about −5 and +5° C., and most preferably lower than about 0° C. In this way, an optimum in selectivity and yield can be achieved.

The ratio of the a-halo ester of formula (6b) to the aromatic aldehyde of formula (6a) may be between about 0.9 and 2, preferably between about 1.0 and 1.5, and more preferably between about 1.0 and 1.2. Similar figures apply for the ratio of the base to the aromatic aldehyde of formula (6a).

The reaction mixture obtained from the process according to Route 6 may be purified by methods known to a person skilled in the art, for example, by neutralization of the excess base, followed by an extraction of water and—optionally—of organic solvent.

### Compound (1)

The present invention relates to a process for preparing the compound (2R,3R)-2-hydroxy-3-amino-3-aryl-propionamide according to formula (1), wherein aryl A is as defined above.

The compound (1) may be obtained according to the process of the present invention in an e.e. of at least about 80%, preferably at least about 85%, more preferably at least about 90%, even more preferably at least about 95%, and most preferably at least about 98%.

The compound (1) may be obtained according to the process of the present invention with a yield of at least about 20%, preferably at least about 30%, more preferably at least about 40%, even more preferably at least about 50%, and most preferably at least about 60%.

With the process of the present invention, compound (1) is obtained in such an acceptable enantiomeric and diastereomeric purity and in such acceptable yields (little by-products), that little or no further purification or recrystallization is required. However, if needed, further purification steps that are common in the art may be applied, for example extraction, filtration, crystallization, distillation or chromatography. A suitable purification may, for example, be a recrystallization from an organic solvent (for example an alcohol, such as methanol, ethanol and the like).

According to a preferred embodiment of the present invention, the aryl A in compound (1) and in the reaction mixture of formula (2) is a phenyl group. In this preferred embodiment, of the four stereoisomers of 2-hydroxy-3-amino-3-phenylpropionamide and the four stereoisomers of 2-amino-3-hydroxy-3-phenylpropionamide that are formed during the reaction, surprisingly the (2R, 3R)-2-hydroxy-3-amino-3-phenyl-propionamide crystallizes and thus can be easily isolated (e.g. by filtration) in relatively high d.e. and e.e., yield and purity.

### Compound 3

According to the process of the present invention, the (2R,3R)-2-hydroxy-3-amino-3-aryl-propionamide (1) may be subsequently converted to the (2R,3R)-2-hydroxy-3-amino-3-aryl-propionic acid alkyl ester of formula (3) above (with R² as defined above) in relatively high yield, d.e. and e.e.

This esterification reaction can, for example, be carried out according to known processes, such as exemplified in Wuts et. al. Tetr.: Asymm. 2000, 11, 2117-2123. Preferably, this esterification process is carried out in the corresponding alkyl alcohol R²—OH (with R² as defined above) in the presence of a strong acid, for instance hydrogen chloride, sulphuric acid, chlorosulphonic acid and the like, or in the presence of an esterification agent, such as thionylchloride, oxalyl chloride and the like. Preferably, the process is carried out in the presence of hydrogen chloride. The organic solvent used in the reaction mixture may or may not be the same as the solvent used in the process to prepare compound (1). The process may be carried out under the conditions known to a person skilled in the art. For example, the corresponding alkyl alcohol may be used as the solvent and thionylchloride may be added drop wise at temperatures below 10° C. or by passing hydrogen chloride gas through the solution. The product may be isolated as a strong acid (preferably HCl) salt directly or after a solvent switch to a suitable solvent. For example, the product can be liberated by extraction with water/dichloromethane at a pH of about 9, and can be isolated from the organic layer.

The invention also relates to the use of the optically active compound according to formula (1) and/or (3) as intermediate in the preparation of pharmaceuticals, and thus, in the framework of the present invention, it is an object to prepare a compound according to formula (1) and (3) in relatively high stereoisomeric purity.

According to the process of the present invention, compound (3) may be further converted into Imidazo[1,2-h][1,7]naphthyridin-7(8H)-one, 5,6,9,10-tetrahydro-8-tert.-butyidimethylsilyloxy-2,3-dimethyl-9-phenyl-, (8R,9R), which may then be converted into Imidazo[1,2-h][1,7]naphthyridin-7(8H)-one, 9, 10-dihydro-8-tert.-butyldimethylsilyloxy-2,3-dimethyl-9-phenyl-, (8R,9R). The latter compound may be subsequently converted into Imidazo[1,2-h][1,7]naphthyridin-7(8H)-one, 9, 10-dihydro-8-hydroxy-2,3-dimethyl-9-phenyl-, (8R,9R), as disclosed for example in WO2004/056362.

Imidazo[1,2-h][1,7]naphthyridin-7(BH)-one, 9, 10-dihydro-8-hydroxy-2,3-dimethyl-9-phenyl-, (8R,9R) may be further converted into a pharmaceutical ingredient or a pharmaceutical active ingredient, in particular into a compound such as for instance, an acid pump antagonist (APA) which is suitable for the treatment of acid-induced gastrointestinal diseases, preferably into (7R,8R,9R)-2,3-dimethyl-8-hydroxy-7-(2-methoxyethoxy)-9-phenyl-7,8,9,10-tetrahydroimidazo-[1,2-h][1,7]naphthyridine, as described in for example WO 03/094967.

Thus, in the process of the present invention, compound (3) may be further converted into a pharmaceutical ingredient or a pharmaceutical active ingredient, in particular into a compound such as for instance, (7R,8R,9R)-2,3-dimethyl-8-hydroxy-7-(2-methoxyethoxy)-9-phenyl-7,8,9,10-tetrahydroimidazo-[1,2-h][1,7]naphthyridine.

The invention will be illustrated with the following examples.

### EXAMPLE 1

### 1.1. Preparation of Ethyl-phenyl-glycidate According to Formula (2)

Ethyl formate (3 g) was added to a solution of sodium ethoxide in ethanol (400g, 20%). The mixture was cooled to 0° C. Benzaldehyde (106 g) was added while keeping the temperature <3° C. Subsequent ethyl-chloro-acetate (130 g) was added in 3 hours while keeping the temperature <1° C. The mixture was aged for 3 hours allowing the temperature to rise to 10° C. After cooling the mixture to <5° C., tri-ethylamine (5 g) and acetic acid (13 g) were added to neutralize the excess base. Water (500 ml) and toluene (250 ml) were added and the mixture was heated to 35° C. After mixing and settling the water phase was separated and once more extracted with toluene (250 ml). The combined toluene layer was concentrated under reduced pressure, yielding 205 g of brownish oil.

GC-analysis: 75% trans- and 2% cis- Ethyl-phenyl-glycidate.

10% toluene

13% other components

Yield=80%

### 1.2. Preparation of a Reaction Mixture According to Formula (2) which is Enriched in Trans-(2R,3S)-ethyl-phenyl-glycidate of Formula (2a)

Water (250 g) and potassium hydrogen carbonate (42 g) were added to 205 g of trans-ethyl-phenyl-glycidate. The mixture was heated to 26° C. A lipase enzyme Novozymes®525 was added and the mixture was stirred for 10 hours. After settling the water phase (which included trans-(2S,3R)-potassium-phenyl-glycidate) was separated. The water layer was extracted once more with toluene (250 ml). The combined toluene layer was filtered over 1 μm filter to remove enzyme. After filtration, the toluene layer was concentrated under reduced pressure yielding 106 g of brownish oil.

GC-analysis: 73% trans- and 3% cis- Ethyl-phenyl-glycidate

10% toluene

14% other components

HPLC analysis: trans-(2R,3S)-ethyl-phenyl-glycidate: ee=86%

Yield=50%.

### 1.3. Preparation of (2R,3R)-2-hvdroxy-3-amino-3-phenylpronionamide According to Formula (1)

106 g of trans-(2R,3S)-Ethyl-phenyl-glycidate (e.e.=86%) was added to a mixture of methanol (150 g) and 25% ammonia (450 g), at 20° C. The mixture was aged for 2 hours (in a closed reactor). After some time trans-(2R,3S)-ethyl-phenyl-glycidamide crystallized. The temperature was increased to 35° C. in 1 hour and the mixture was aged for 16 hours. (2R, 3R) 2-hydroxy-3-amino-3-phenylpropionamide crystallized out. After cooling to 20° C. in approx. 3 hours, (2R, 3R) 2-hydroxy-3-amino-3-phenylpropionamide was filtered off and washed with ethanol. (2R, 3R) 2-hydroxy-3-amino-3-phenylpropionamide was dried at max 40° C. under vacuum, yielding 43 g of white crystalline product.

HPLC: e.e. >99.5%

HPLC: assay >98%

¹H NMR (DMSO-d6) δ: 1.96 (bs, 2), 3.97 (m,1), 4.05 (d,1, J=2.4), 5.41 (d,1, J=2.5), 7.03 (bs, 2), 7.16-7.31 (m, 5)

Yield=60% - Overall yield from benzaldehyde=23%

### EXAMPLE 2

### 2.1. Preparation of Ethyl-phenyl-glycidate

This preparation is identical to Example 1.1.

### 2.2. Preparation of a Reaction Mixture According to Formula (2) which is Enriched in Trans-(2R.3S)-ethyl-phenyl-glycidate According to Formula (2a)

Water (250 g) and potassium hydrogen carbonate (42 g) were added to 205 g of trans-ethyl-phenyl glycidate. The mixture was heated to 26° C. A lipase enzyme Novozymes®525 was added and the mixture was stirred for 12 hours. After settling the water phase (which included trans-(2S,3R)-potassium-phenyl-glycidate) was separated. The water layer was extracted once more with toluene (250 ml). The combined toluene layer was filtered over a 1 μm filter to remove enzyme. After filtration the toluene layer was concentrated under reduced pressure yielding 101 g of brownish oil.

GC-analysis: 73% trans- and 3% cis- Ethyl-phenyl-glycidate

10% toluene

14% other components

HPLC analysis: trans-(2R,3S)-ethyl-phenyl-glycidate: ee=89%

Yield=48%.

### 2.3. Preparation of (2R,3R) 2-hydroxy-3-amino-3-phenylpropionamide According to Formula (1)

101 g of trans-(2R,3S)-Ethyl-phenyl-glycidate (e.e.=89%) was added to a mixture of methanol (150 g) and 25% ammonia (450 g) at 20° C. The mixture was aged for 2 hours (in a closed reactor). After some time trans-(2R,3S)-ethyl-phenyl-glycidamide crystallized. The temperature was increased to 35° C. in 1 hour and the mixture was aged for 16 hours. (2R,3R) 2-hydroxy-3-amino-3-phenylpropionamide crystallized. After cooling to 10° C. in approx. 3 hours, (2R, 3R) 2-hydroxy-3-amino-3-phenylpropionamide was filtered off and washed with ethanol. (2R,3R) 2-hydroxy-3-amino-3-phenylpropionamide was dried at max 40° C. under vacuum, yielding 41.5 g white crystalline product.

HPLC: e.e. >99.5%

HPLC: assay >98%

¹H NMR (DMSO-d6) δ: 1.96 (bs, 2), 3.97 (m,1), 4.05 (d,1, J=2.4), 5.41 (d,1, J=2.5), 7.03 (bs, 2), 7.16-7.31 (m, 5)

Yield=61% - Overall yield from benzaldehyde=23%

### EXAMPLE 3

### 3.1. Preparation of Ethyl-phenyl-glycidate According to Formula (2)

Ethyl formate (3 g) was added to a solution of potassium ethoxide in ethanol (400 g, 20%). The mixture was cooled to 0° C. Benzaldehyde (106 g) was added while keeping the temperature <3° C. Subsequently ethyl chloroacetate (130 g) was added in 3 hours while keeping the temperature <1° C. The mixture was aged for 3 hours, allowing the temperature to rise to 10° C. After cooling the mixture to <5° C, tri-ethylamine (5 g) and acetic acid (13 g) were added to neutralize the excess base. Water (500 ml) and toluene (250 ml) were added and the mixture was heated to 35° C. After mixing and settling, the water phase was separated and once more extracted with toluene (250 ml). The combined toluene layer was concentrated under reduced pressure, yielding 205 g of brownish oil.

GC-analysis: 77% trans- and 3.8% cis- Ethyl-phenyl-glycidate.

10% Toluene

9% other components

Yield=82%

### 3.2. Preparation of a Reaction Mixture According to Formula (2) which is Enriched in trans-(2R,3S)-ethyl-phenyl-glycidate According to Formula (2a)

Water (250 g) and potassium hydrogen carbonate (42 g) were added to 205 g trans-ethyl-phenyl-glycidate. The mixture was heated to 26° C. A Novozymes®525 lipase enzyme was added and the mixture was stirred for 8 hours. After settling the water phase (which included trans-(2S,3R)-potassium-phenyl-glycidate) was separated. The water layer was extracted once more with toluene (250 ml). The combined toluene layer was filtered over a 1 μm filter to remove enzyme. After filtration the toluene layer was concentrated under reduced pressure, yielding 106 g of brownish oil.

GC-analysis: 75% trans- and 7.3% cis- ethyl-phenyl-glycidate

8% toluene

10% other components

HPLC analysis: trans-(2R,3S)-ethyl-phenyl-glycidate: ee=84%

Yield=51%.

### 3.3. Preparation of (2R,3R)-2-hydroxy-3-amino-3-phenylpropionamide According to Formula (1)

106 g of trans-(2R,3S)-ethyl-phenyl-glycidate (e.e.=84%) was added to a mixture of methanol (150 g) and 25% ammonia solution (450 g) at 20° C. The mixture was aged for 2 hours (in a closed reactor). After some time trans-(2R,3S)-ethyl-phenyl-glycidamide crystallized. The temperature was increased to 35° C. in 1 hour and the mixture was aged for 16 hours. (2R, 3R) 2-hydroxy-3-amino-3-phenylpropionamide crystallized. After cooling to 20° C. in approx. 3 hours, (2R, 3R) 2-hydroxy-3-amino-3-phenylpropionamide was filtered off and washed with ethanol. (2R,3R) 2-hydroxy-3-amino-3-phenylpropionamide was dried at max. 40° C. under vacuum, yielding 41.6 g of white crystalline product.

HPLC: e.e. >99.5%

HPLC: assay >98%

¹H NMR (DMSO-d6) 6: 1.96 (bs, 2), 3.97 (m,1), 4.05 (d,1, J=2.4), 5.41 (d,1J=2.5), 7.03 (bs, 2), 7.16-7.31 (m, 5)

Yield=59% - Overall yield from benzaldehyde=24%

### EXAMPLE 4

### Preparation of (2R,3R)-2-hydroxy-3-amino-3-phenyl-propionic acid ethyl ester According to Formula (3) wherein R² is Ethyl and A is Phenyl

25 g of (2R,3R)-2-hydroxy-3-amino-3-phenylpropionamide was added to a mixture of ethanol (200 g) and thionyl chloride (24 g) at 20° C. The mixture was aged to reflux. Thionyl chloride (42 g) was added over a period of 5 hours. After aging for 10 hrs the reaction was completed (98% conversion). Ethanol was distilled off until 75 ml residue volume. Toluene was added at 80° C. The mixture was cooled to 10° C. The solids were filtered off and washed with 100 ml of toluene. The solids were dried under vacuum, yielding 41.7 g of white solid (mixture of product and ammonium chloride).

HPLC: assay=78% (2R,3R)-2-hydroxy-3-amino-3-phenyl-propionic acid ethyl ester hydrochloric acid salt

Yield=97%

1. Process for the preparation of (2R,3R)-2-hydroxy-3-amino-3-aryl-propionamide according to general formula (1), wherein aryl A represents a substituted or unsubstituted aromatic ring, 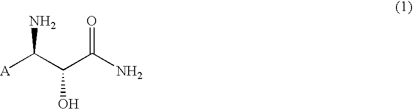
in which a reaction mixture comprising the two enantiomers (2R,3S) and (2S,3R) of trans-3-ary\ glycidic acid alkyl ester and the two enantiomers (2R,3R) and (2S,3S) of cis-3-aryl glycidic acid alkyl ester, said ester being represented by the general formula (2), wherein R¹ is an ester residue which may be an optionally substituted alkyl, cycloalkyl, aryl, aralkyl or alkaryl group, 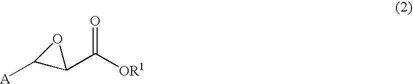
is reacted with ammonia, and wherein the reaction mixture is enantiomerically and diastereomerically enriched in the (2R,3S)-trans-3-arylglycidic acid alkyl ester.

2. Process according to claim 1, wherein the reaction mixture is enantiomerically and diastereomerically enriched in the (2R,3S)-trans-3-arylglycidic acid alkyl ester in an e.e. of at least about 40% and a d.e. of at least about 30%.

3. Process according to claim 1 wherein the reaction mixture is enantiomerically and diastereomerically enriched in the (2R.3S)-trans-3-arylglycidic acid alkyl ester in an e.e. of at least about 50% and a d.e. of at least about 50%.

4. Process for the preparation of (2R,3R)-2-hydroxy-3-amino-3-aryl-propionamide of formula (1) according to any one of claims 1-3, wherein the reaction mixture which is enantiomerically and diastereomerically enriched in the (2R,3S)-trans-3-arylglycidic acid alkyl ester is prepared by stereoselective hydrolyzing a reaction mixture comprising the four enantiomers (2R,3S) and (2S,3R) of trans-3-aryl glycidic acid alkyl ester and (2R,3R) and (2S,3S) of cis-3-aryl glycidic acid alkyl ester of the compound according to formula (2) by using an enzyme originating from Candida Antarctica.

5. Process according to claim 4, wherein the (2S,3R)-trans-3-arylglycidic acid alkyl ester is stereoselective hydrolyzed.

6. Process according to claim 4, wherein the non-hydrolyzed (2R,3S)-trans-3-arylglycidic acid alkyl ester is not separated off from the reaction mixture.

7. Process according to claim 4, wherein the reaction mixture comprising the four enantiomers of the compound according to formula (2) is prepared by contacting an aromatic aldehyde according to formula (6a), 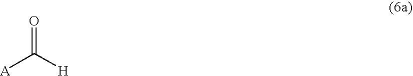
wherein A represents a substituted or unsubstituted aromatic ring, with an α-halo ester according to formula (6b) 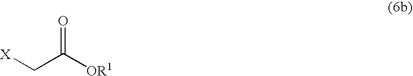
wherein X is selected from Cl, Br, F or I, and wherein R¹ is as defined above, in the presence of a base.

8. Process according to claim 7, wherein the α-halo ester is α-chloroacetic acid methyl ester or a-chloroacetic acid ethyl ester and wherein the base is an alkoxide of an alkaline earth metal.

9. Process according to claim 9, wherein the process is carried out in the presence of an alcohol.

10. (2R,3R)-2-hydroxy-3-amino-3-aryl-propionamide according to formula (1), 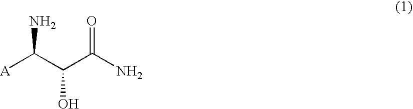
in which aryl A represents a substituted or unsubstituted aromatic ring.

11. (2R,3R)-2-hydroxy-3-amino-3-phenyl-propionamide according to formula (1), wherein aryl A represents a phenyl group.

12. Process for the preparation of (2R,3R)-2-hydroxy-3-amino-3-aryl-propionic alkyl ester according to the general formula (3), 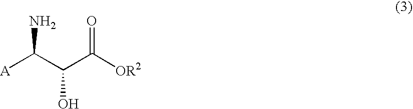
in which A represents a substituted or unsubstituted aromatic ring and alkyl R² represents a substituted or unsubstituted alkyl group with 1-10 carbon atoms, wherein (2R,3R)-2-hydroxy-3-amino-3-aryl-propionamide according to formula (1) is contacted with an alkyl alcohol R²—OH in the presence of a strong acid or an esterification agent.

13. Process according to claim 1, wherein the obtained compound (1) or (3) is further converted into imidazo[1,2-h][1,7]naphthyridin-7(8H)-one, 9,10-dihydro-8-hydroxy-2,3-dimethyl-9-phenyl-, (8R,9R) in a manner known per se.

14. Process according to claim 1, wherein the obtained compound is further converted into (7R,8R,9R)-2,3-dimethyl-8-hydroxy-7-(2- methoxyethoxy)-9-phenyl-7,8,9,10-tetrahydroimidazo-[1,2-h][1,7]naphthyridine in a manner known per se.

15. Use of the compound (1) or (3) obtained by a process according to claim 1 in the preparation of imidazo[1,2-h][1,7]naphthyridin-7(8H)-one, 9,10-dihydro-8-hydroxy-2,3-dimethyl-9-phenyl-, (8R,9R).

16. Use of a compound obtained by a process according to claim 1 in the preparation of (7R,8R,9R)-2,3-dimethyl-8-hydroxy-7-(2-methoxyethoxy)-9- phenyl-7,8,9,10-tetrahydroimidazo-[1,2-h][1,7]naphthyridine.
